(19) United States
(12) Patent Application Publication
(10) Pub. No.: US 2020/0360036 A1
(43) Pub. Date: Nov. 19, 2020

(54) Mastopexy clamp

(71) Applicant: Edward Domanskis, Newport Beach, CA (US)

(72) Inventor: Edward Domanskis, Newport Beach, CA (US)

(21) Appl. No.: 16/501,618

(22) Filed: May 13, 2019

(51) Int. Cl. A61B 17/28

(52) U.S. Cl. CPC A61B 17/282; A61B 2017/2837; A61B 17/2833; A61B 17/2841

(57) ABSTRACT

A surgical forceps for breast lift procedures has a pair of intersecting arms mounted to each other for scissor-like pivotal movement relative to each other, each arm having a handle portion and a clamp portion, each handle portion including a finger loop and a ratchet lock element for releasably engaging an opposing cooperating element on the other handle portion. The clamp portion of each arm provides an elongate gripping surface, and from the distal ends of each clamp portion extends an elongate foot bar at about 90 degrees to the gripping surface, the bars combining to provide a generally straight lower edge.

The forceps can be manipulated from an open position, to a clamping position where the opposing gripping surfaces engage each other, and the foot bars extend oppositely forming an inverted-T configuration with the clamp portions, and the ratchet lock elements releaseably engage each other. Skin tissue engaged by the gripping surfaces can advantageously be manipulated as desired.

### BACKGROUND OF THE INVENTION

### 1. Field of the Invention

The present invention relates generally to surgical clamps, and more particularly to forceps for clamping and repositioning skin tissue in mastopexy and breast-lift procedures.

### 2. Description of the Prior Art

Mastopexy is a surgical procedure performed to correct breast ptosis (sagging), which in the broadest sense stems from a discrepancy between the volume of underlying breast tissue and the surface area of the surrounding skin envelope or some combination thereof. The goals of Mastopexy include restoring the normal contours of the breast mound. It can involve removal and tightening of excess breast skin as well as repositioning of the nipple areola complex (NAR).

Common lift procedures include the Crescent, Vertical and the Inverted-T or Anchor technique.

In the Inverted-T technique, for example, the operator makes lines above the upper edge of the NAR marking its new location, and below the NAR. Skin in these areas will be separated from tissue below and removed. Once excess skin is removed, new skin edges are pulled together and sutured or stapled in place. The surgeon will use best estimates in carrying out these steps. The NAR can be moved to the desired new location and similarly affixed.

Sometimes during such surgery the vertical incision undesirably extends into the patient's abdominal region.

There remains is a need for performing breast lift procedures with greater simplicity, accuracy and quickness.

### BRIEF SUMMARY OF THE INVENTION

In view of the foregoing, it is an object of the present invention to provide an instrument for performing breast uplift surgery with improved quickness and accuracy.

Accordingly, the present invention provides surgical forceps having first and second elongate arms pivotally connected in a scissors fashion, each arm having a handle portion and an elongate clamp portion, the handle portions lying in substantially the same plane. The handle portions have finger and thumb loops and a ratchet mechanism for releasably latching these portions. The clamp portions each provide an elongate generally planar gripping surface, and feature an elongate foot bar extending outwardly from distal end of each clamp portion at about 90 degrees to the plane of the gripping surface. A further feature is the curvature of the clamp portions in a plane perpendicular the plane of the handle portions.

When the forceps are manipulated from on open position to a clamp position the elongate gripping surfaces are brought towards engagement with each other and the ratchet mechanism will be engaged so as to releasably lock the forceps. The foot bars are aligned and combine to provide a generally straight lower edge. This facilitates as a guide for proper length and direction of incisions.

In use, breast tissue on opposing sides of a line of a straight incision can be grasped to tighten the breast envelope, the clamp portions following the curvature of the breast, and the foot bars engaging the breast surface.

The present invention will be further appreciated in view of the drawings, detailed description, and claims, which follow.

### BRIEF DESCRIPTION OF THE DRAWINGS

FIG. 1 is a perspective view of a mastopexy clamp according to the present invention;

FIG. 2 is a rear elevational view of the clamp shown in FIG. 1 in closed position;

FIG. 3 is a left side elevation view of the clamp of FIG. 1; and

FIG. 4 is a right side elevational view of the clamp.

### DETAILED DESCRIPTION OF THE INVENTION

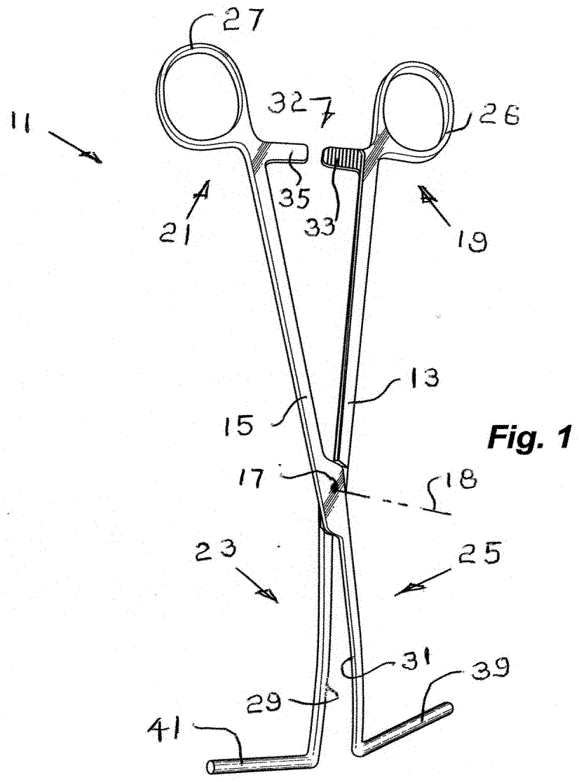

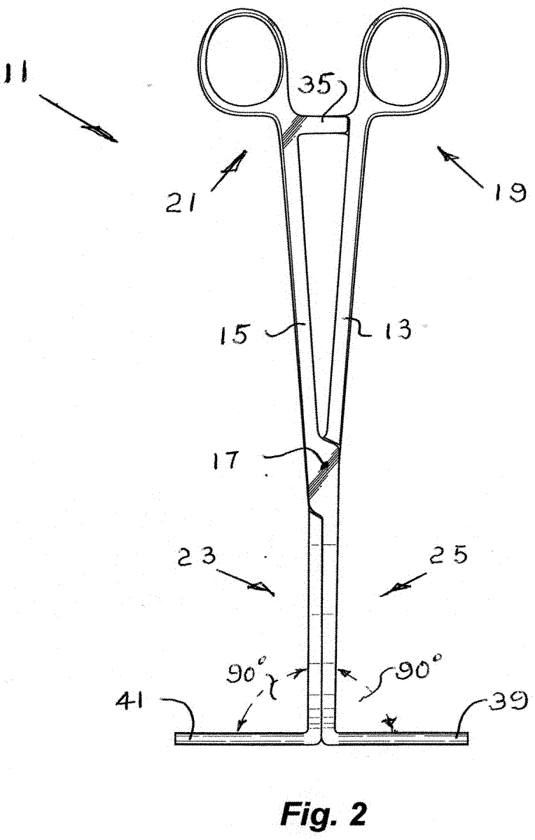

Referring now to the drawings, FIG. 1 shows surgical forceps 11 according to the present invention having first and second arms 13 and 15 pivoted in scissors fashion about pivot pin or screw 17, for rotation about axis 18. Arms 13 and 15 have handle portions 19 and 21, and clamp portions 23 and 25. Handle portions 19 and 21 include finger/thumb loops 26 and 27, respectively. There is locking mechanism 32 comprising a ratchet arm 33 and an opposing ratchet catch 35 that lie in substantially the same plane. The clamp portions 23 and 25 are elongate and feature gripping surfaces 29 and 31 that lie in opposition to each other and will engage one another when the forceps are rotated to a closed position, shown in FIG. 2. Surface 29 (not shown) mirrors the surface 31 and is generally planar. In variants of the invention these surfaces can be provided with gripping enhancements such as grid patterns of intersecting peaks and channels.

Elongate foot bars 39 and 41, have generally circular cross-sectional configurations, and extend from the distal ends of clamp portions at about 90° to the clamp portions, as best shown in FIG. 2, and align with each other when in the closed position shown, and combine to provide a generally straight lower edge.

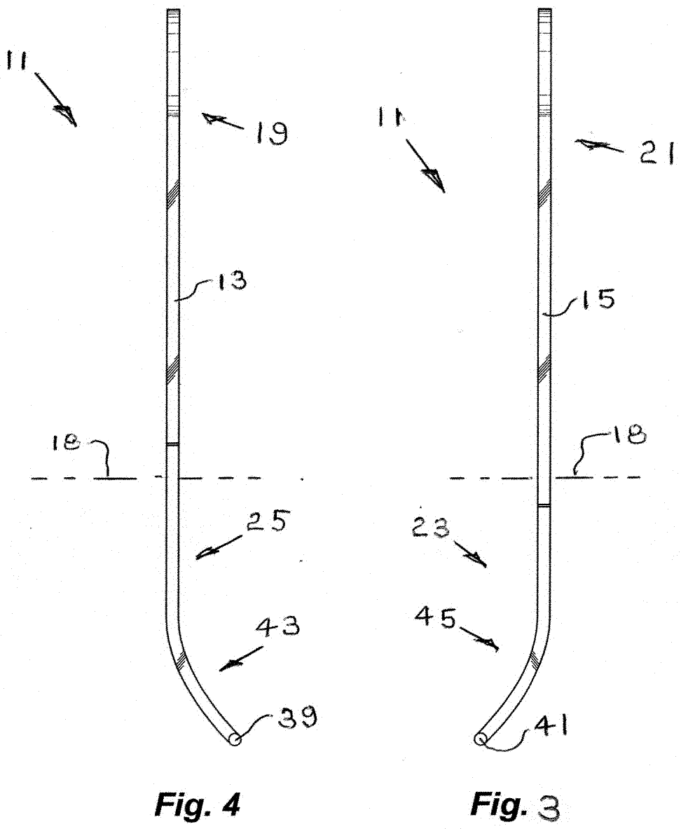

FIG. 3 best shows that a substantial part of the clamp portions 23 and 25 are curved at 43 and 45. In variants of the invention the degree and length of curvature of these portions can vary.

Referring now to the ratchet mechanism 31, it should be appreciated that when an operator manipulates the handle portion to move the forceps from the open position as shown in FIG. 1 towards a clamp position shown in FIG. 2, the ratchet arm 33 will engage the ratchet catch 35. This will prevent the handle portions 19 and 21, and the clamp portions 23 and 25 from separating. The forceps can be unlatched by the operator, in a known manner, by apply a twisting movement in order to lift the ratchet catch 35 away from the ratchet arm 33.

The forceps 11 can be used advantageously in breast lift surgery to firmly grip skin tissue on opposing sides of a straight longitudinal incision in the breast, with the curved portions following the curvature of the engaged breast.

Forceps 11 are fabricated of high-grade carbon steel or other suitable high-quality alloys using know techniques in the industry.

Various modifications and variations of the invention will be evident to persons of ordinary skill in the art, given the benefit of this disclosure, and it is intended that the invention be given its full scope and breath as defined in the claims that follow.

What is claimed is:

1. A surgical forceps for clamping skin tissue in breast-lift surgery, comprising:
a) first and second elongate arms pivotally connected in a scissors fashion, each said arms having a handle portion, and an elongate clamp portion, said handle portions lying in substantially the same plane;
b) said handle portions each having a finger loop and ratchet means for making releaseable locking engagement with ratchet means on the other handle portion;
c) the clamp portion of each of said arms having an elongate gripping surface, lying in a plane;
d) elongate foot bar extending outwardly from the distal ends of each clamp portion at about 90 degrees to the plane of said gripping surface;
e) said gripping surfaces being operably opposed and said latching means making releasable engagement when said handle portions are rotated towards each other for locking said forceps in a fixed position; and
f) wherein the said elongate clamp portion is curved in a plane perpendicular to the plane of said handle portions.

2. Forceps as defined in claim 1 wherein said gripping surfaces are generally flat.

3. Surgical forceps as defined in claim 1 wherein said gripping surfaces contain a grid pattern of intersecting peaks and channels.

4. Forceps as defined in claim 1 wherein said bars have a generally circular cross-section.
